(19) United States
(12) Patent Application Publication
(10) Pub. No.: US 2025/0137033 A1
(43) Pub. Date: May 1, 2025

(54) DNA UNFOLDING USING A FREE-END TAG FLOW MODIFIER

(71) Applicant: Robert Bosch GmbH, Stuttgart (DE)

(72) Inventors: Karim Gadelrab, Watertown, MA (US); Christian Grumaz, Waiblingen (DE); Nadezda Fomina, Redwood City, CA (US); Christopher Johnson, Sunnyvale, CA (US); Sebastian Pilsl, Rutesheim (DE)

(73) Assignee: Robert Bosch GmbH, Stuttgart (DE)

(21) Appl. No.: 18/499,328

(22) Filed: Nov. 1, 2023

(51) Int. Cl. C12Q 1/6806

(52) U.S. Cl. CPC C12Q 1/6806

(57) ABSTRACT

Methods for leveraging the charged nature of DNA to promote DNA extension and manipulate its orientation are provided. The methods include attaching highly charged molecules to a free end of a DNA molecule to transform the DNA molecule into a pseudo dipole. Upon applying an external electric field, the charge separation may cause the DNA to extend and/or to align with the electric field lines. This approach offers a practical means to achieve a controlled, unfolded state of DNA with a predetermined orientation.

### TECHNICAL FIELD

In at least one aspect, the present disclosure relates to methods for unfolding DNA.

### BACKGROUND

Deciphering the genetic information encoded in DNA molecules necessitates the capability to induce and to maintain a transition from the natural folded structure to a less favorable extended state. Utilizing nanoscale confinement and electrophoretic forces holds promise in facilitating DNA unfolding. Efficiently guiding DNA into confined spaces without generating excessive forces that accelerate the DNA and thus compromise sensing and mapping applications, however, remains a challenge. A core issue is a limited ability to apply non-uniform forces along the DNA axis to promote stretching and unfolding. There is a need for methods that efficiently promote DNA extension and manipulate its orientation.

### SUMMARY

In various aspects, a method for manipulating the conformation and/or orientation of a DNA molecule is provided. The method comprises obtaining a DNA molecule; modifying an end of the DNA molecule by coupling a charged molecule to the end of the DNA molecule; applying an external force to the DNA molecule; and adjusting the strength of the external force to induce the DNA molecule to change conformation and/or orientation.

In other aspects, a method for unfolding a DNA molecule is provided. The method comprises obtaining a DNA molecule; modifying an end of the DNA molecule by coupling a charged molecule to the end of the DNA molecule; and subjecting the DNA molecule to an electric field.

In yet other aspects, a method for orienting a DNA molecule is provided. The method comprises obtaining a DNA molecule; modifying an end of the DNA molecule by coupling a polyionic tag to the end of the DNA molecule to create a pseudo-dipole state; subjecting the DNA molecule to an electric field; and adjusting the strength of the electric field to induce the DNA molecule to align with an axis of the electric field.

### BRIEF DESCRIPTION OF THE DRAWINGS

FIG. 1A illustrates a single stranded DNA molecule (ssDNA) with a drag tag subjected to an electric field.

FIG. 1B illustrates normalized end-to-end distance to max DNA length <R>/Lmax for ssDNA for different magnitudes of force E×q obtained from molecular dynamics simulations.

FIG. 1C illustrates a double stranded DNA molecule (dsDNA) with a drag tag subjected to an electric field.

FIG. 1D illustrates normalized end-to-end distance to max DNA length <R>/Lmax for dsDNA for different magnitudes of force E×q obtained from molecular dynamics simulations.

FIG. 2A illustrates a double stranded DNA molecule (dsDNA) with a drag tag subjected to an electric field.

FIG. 2B illustrates employing drag tags for gaining control over dsDNA orientation.

FIG. 2C illustrates a single stranded DNA molecule (ssDNA) with a drag tag subjected to an electric field.

FIG. 2D employing drag tags for gaining control over ssDNA orientation.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “complementarity”, “complementary” or “complement” may refer to the ability of a nucleic acid to form hydrogen bond(s) with another nucleic acid sequence by either traditional Watson-Crick or other non-traditional types.

The phrase “conformational change” when referring to a DNA molecule may be used to describe a change in the spatial configuration of a DNA molecule including bending or stretching the DNA molecule or altering its twist, writhe, or linking number.

The terms “unfolding”, “stretching”, “extending”, or “extension” when used in reference to DNA may be used interchangeably to describe a conformational change in a DNA molecule that results in the DNA molecule moving toward a more linear conformation.

The terms “charged molecule”, “tag” and “drag tag” may be used interchangeably throughout this disclosure.

The terms “highly charged” or “super charged” may be used interchangeably throughout this disclosure and refer to molecules with a magnitude greater than the negative linear charge density of DNA. The linear charge density of DNA refers to the amount of electric charge in a line of DNA. The linear charge density of DNA may vary according to the molar concentration of counterions. Counterions are positively charged ions to which DNA is exposed along with water molecules under physiological conditions. A super or highly negatively charged molecule refers to a molecule with greater than or equal to −2e/molecule over the linear charge density of the DNA molecule to which is it coupled. A super or highly positively charged molecule refers to a molecule with greater than or equal to 1e/molecule over the linear charge density of the DNA molecule to which it is attached. “e” refers to elementary charge, which is the charge on a single proton or electron (1.602176487(40)×10⁻¹⁹ Coulombs).

Throughout this application, where publications are referenced, the disclosures of these publications in their entireties are hereby incorporated by reference into this application to more fully describe the state of the art to which this invention pertains.

The DNA molecule contains the genetic information that governs the development of cells in all living organisms. This pivotal function underlies the aspiration to realize personalized medicine and targeted therapies. An understanding of the specific sequences of the genes and gene mutations involved in causing disease states can potentially be utilized to create individualized therapeutics and also to answer fundamental inquiries in biology.

In its native form, DNA undergoes significant conformational changes, including bending and twisting, which lead to a densely packed structure capable of fitting into a tiny cell or cell nucleus. This inherent folding of DNA presents challenges in accessing the encoded genetic information for mapping and sequencing purposes. Unfolding DNA is therefore crucial for the analysis of DNA sequences and structures and the utilization of genetic information in biological and medical applications. Various techniques for unfolding or manipulating the orientation of DNA molecules have been proposed in the prior art, including hydrodynamic forces, optical tweezers, and Nano-confinement. These techniques have had varying degrees of success and complexity.

A key to unfolding DNA lies in the ability to apply non-uniform forces along the DNA chain, causing different segments of the molecule to displace relative to each other. Simply applying electrostatic forces to native DNA may be insufficient due to the uniform charge distribution of the molecule and the constant charge-to-friction ratio during free solution electrophoresis. However, by chemically attaching charged end-tags to the DNA molecule, a scenario may be created in which super-negatively or positively charged ends experience different forces (proportional to the sign and magnitude of the charge) under the influence of an external electric field.

The concept of DNA mobility modifiers has been explored to achieve size-based separation of DNA in free-solution electrophoresis. This approach involves using drag tags, which can be either neutral or charged, acting as hydrodynamic parachutes that slow down small DNA molecules compared to larger ones. By manipulating the chemistry of DNA ends, it becomes possible to control DNA unfolding and orientation in a relatively straightforward and accessible manner.

In one or more embodiments provided herein, methods are disclosed for leveraging the charged nature of DNA to promote DNA extension and to manipulate its orientation. The method according to at least one embodiment includes attaching highly charged molecules (positive or negative) referred to as “tags” or “drag tags” to one of the free ends of a DNA molecule to transform the DNA molecule into a pseudo dipole. Upon applying an external electric field, the charge separation may cause the DNA to extend and align with the electric field lines. This approach offers a practical means to achieve a controlled, unfolded state of DNA with a predetermined orientation.

The method of one or more embodiments may include chemically altering one or both ends of a DNA molecule to add super negatively charged or positively charged molecules. Applying an external electric field to the chemically altered DNA molecule may generate a non-uniform force along the DNA axis due to the creation of a pseudo-dipole state. With the correct chemical functionalization and electric field magnitude, DNA unfolding may be achieved in a relatively straightforward manner.

According to certain embodiments, chemically altering an end of a DNA molecule may include chemically attaching a tag (e.g., a “drag-tag”) to the end of the DNA molecule. Examples of suitable drag-tags include but are not limited to polyanionic tags or polycationic tags. Polyanionic tags may include acrylamide copolymers, alginates, lignin sulfonates, pectins, poly-acrylic acid and co-polymers thereof, poly-vinyl sulfuric acid, poly-carbonic acids, polysaccharides, or poly-styrene sulfonic acid. Polycationic tags may include phenols, poly-allylamine, poly(4-vinylbenzylthrimethyl-ammonium) salts, poly-diallyldimethyl-ammonium sales, poly-ethylenimine, poly-vinylamine, poly-vinylpyridine, poly-vinylammonium salts, spermines, or spermidines, peptide sequences (e.g., poly-lysine), aminoplasts (e.g., melamine resins), or polyamidoamines (dendrimers).

The drag-tags may be of linear, branched or cross-linked morphology, depending on the scope of the application. Dendrimers may be fine-tuned in terms of size, charge, and three-dimensional branching. This feature may enable fine-tuning of speed and alignment to regulate how fast the DNA wanders through constrained space for example.

The drag-tags may additionally be functionalized with various functional groups that enable coupling to modified or unmodified DNA. For example, the drag-tags may be modified with residues enabling bioorthogonal click chemistry, such as alkyne, azide, trans-cyclooctene, tetrazine, tetrazole, azirine, sydnone, alkene, oxime, protected alkohyamines, or aldehydes; with alkenes, alkynes, amides or thiols; biotin or streptavidin; phosphoroamidites enabling solid phase synthesis, for example in conjunction with DNA; activated esters (succinimidyl ester) and amines; or aryl halides, boronic esters, thiols, and alkenes for metal-catalyzed carbon-carbon bond formation.

The above-mentioned strategies to couple the drag tag of interest to a DNA molecule may be used for direct coupling of the drag tag to DNA. As an alternative, a drag tag may be indirectly coupled to a DNA molecule via PCR, hybridization, or ligation of an oligonucleotide conjugated to a drag tag that is reverse complementary to the DNA of interest. These conjugates may serve as primers or unique molecular identifiers (UMIs), depending on their application. In such cases, the conjugation can be realized via solid phase synthesis as an example. The conjugated oligonucleotide may be composed of natural nucleic acids or modified nucleic acids. Modified nucleic acids may be used for example to increase the melting temperature of the hybridization or to modulate the stiffness of the double strand. Suitable modified nucleic acids include, for example, phosphorothioate nucleic acids (PNAs), zwitterionic nucleic acids (ZNAs), locked nucleic acids (LNAs), etc.

In other embodiments, a polyionic tag may be attached to a DNA molecule by first introducing an initiator or a chain transfer moiety to the end of the DNA molecule via one of the chemistries described above, and then adding monomers to form a polymer chain.

In yet other embodiments, in addition to stretching a molecule of DNA by adding drag tags to one or both ends, molecules that bind to internal locations on the DNA molecule may be added. For example, single binding proteins, such as gp32 and recA, may unwind single stranded DNA. Like dendrimers, these rather large protein tags can be used to control the number of DNA molecules which are populating a constrained space at the same time. Furthermore, these proteins can guide the path on which the DNA translocates through the electrostatic field, especially in the case of constrained space. Additionally, these large tags might be used to finetune the speed at which the DNA is wandering in an electrostatic field.

DNA molecules may be obtained by isolation from any cell, tissue, or organism or generated from a DNA or RNA molecule from any cell, tissue, or organism via PCR techniques for example. DNA molecules may also be obtained from clinical or environmental samples including skin swabs, bodily fluid samples, or swabs of environmental surfaces for example. Alternatively, DNA molecules may be synthetically generated by oligonucleotide synthesis for example. The DNA molecules may be single or double stranded and may include modified nucleotides. The DNA molecules may range in length from about 200 to about 500 base pairs.

The DNA molecule to be manipulated may be delivered to a device. The device may have an area containing a semi solid or non-solid substance to which the DNA molecule may be delivered and through which the DNA molecule may move. The device may include at least two electrodes spaced at a distance from one another with the semi solid or non-solid substance occupying the space between the electrodes. When activated, the electrodes may produce an electric field.

A controller may be utilized to implement the methods described herein. For example, the controller may actuate delivery of the semi solid or non-solid substance to the device and actuate delivery of the DNA molecule to the substance. The controller may also activate the electrodes to produce the electric field.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

### EXAMPLES

Molecular dynamics simulations were employed to assess the viability of the drag tag strategy in achieving DNA extension. The magnitude of forces and external field were evaluated and correlated to the degree of DNA extension. The DNA molecules utilized in the simulations were around 250 base pairs.

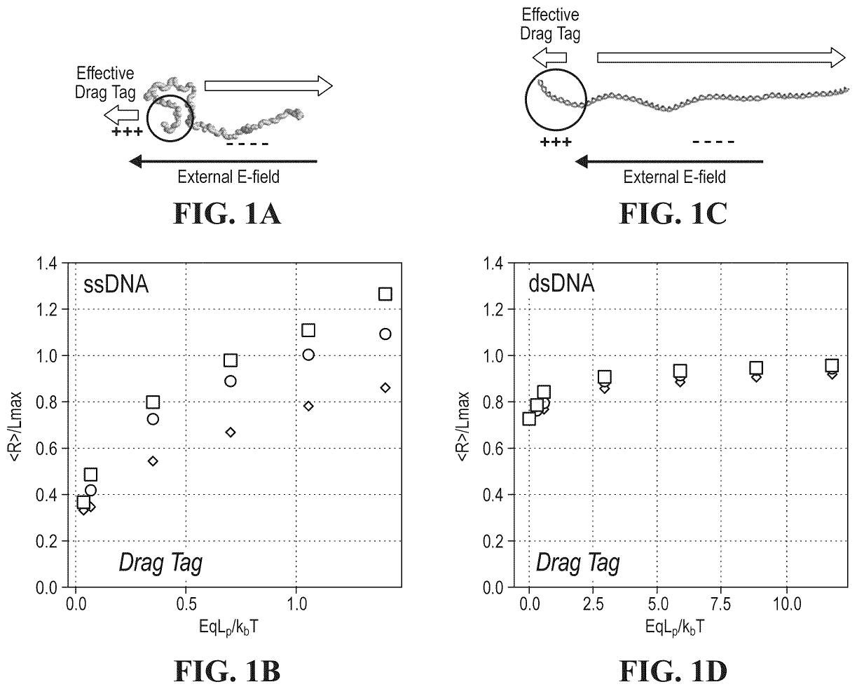

FIGS. 1A-D illustrate the behavior of DNA molecules under extensible force obtained from molecular dynamics simulations. Without loss of generality, a positive tag attached to a single free end of a DNA molecule is simulated. FIG. 1A illustrates a ssDNA molecule with a tag. FIG. 1B shows the results of the simulation for DNA molecules such as that shown in FIG. 1A. FIG. 1C illustrates a dsDNA molecule with a tag. FIG. 1D shows the results of the simulation for DNA molecules such as that shown in FIG. 1C. Overall, results from the simulations shown in FIGS. 1B and 1D are transferable to a situation in which tags are utilized to create two oppositely charged ends, or to couple a super negative drag tag at one end. Results are also transferable to DNA molecules with lengths from about 200 to about 10,000 base pairs. Referring now to FIGS. 1B and 1D, under the application of an external field, the overall length of the DNA molecule increases compared to the no-Field state. This is evaluated by observing the evolution of the average end-to-end distance <R> of the DNA. To ensure generality of the plots, the magnitude of <R> is normalized by the max DNA length (Lmax). The extent of DNA extension is strongly dependent on the nature of the DNA molecule (e.g., single-stranded DNA (ssDNA) or double-stranded DNA (dsDNA)). The rigid dsDNA (characterized by a large persistence length Lp) is already ˜70% of Lmax without external force. The increase of the electric field (E) caused the DNA molecule to reach ˜95% Lmax. The strategy of drag tag is more relevant to the ssDNA which is significantly softer than dsDNA and curls to ˜35% L-max at no external force. Hence, a substantial increase in <R> is observed under the application of external force. The magnitude of the force can be controlled in different manners, including by increasing the magnitude of the E field, increasing the drag tag charge (q), or increasing the total number of molecules constituting the drag tag (represented by the difference in marker size in the plots of FIGS. 1B and 1D). The master plots in FIGS. 1B and 1D highlight the feasibility of the disclosed engineering design of the drag tags and the external operating conditions to achieve DNA unfolding.

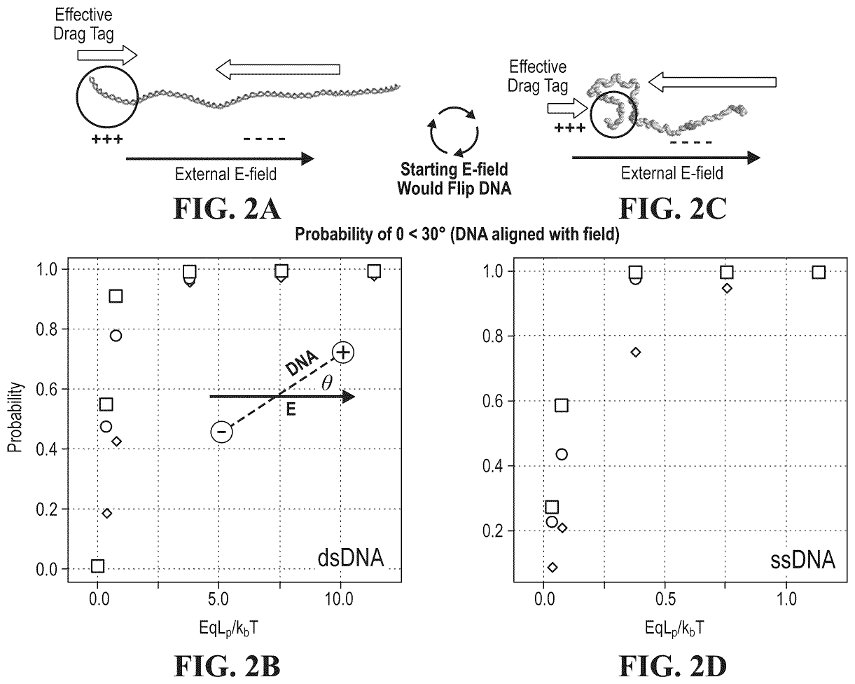

In addition to attaining an unfolded state of a DNA molecule, the drag tag strategy may provide control over DNA orientation. This is a result of the pseudo-dipole state that the DNA would be in due to the separation of charges. Thermodynamically, the formed dipole would fluctuate with its long axis aligned with the E-field lines, and the more positively charged end pointing in the field direction. FIG. 2A illustrates a dsDNA molecule with a tag. FIG. 2B shows results from a simulation with DNA molecules such as that shown in FIG. 2A. FIG. 2C illustrates a ssDNA molecule with a tag. FIG. 2D shows results from a simulation with DNA molecules such as that shown in FIG. 2C. FIGS. 2B and 2D show the probability of having the DNA axis aligned with the field axis (within an arbitrary angle of 30°). The DNA molecule is subjected to an external force. Adjusting the magnitude of the external force to which the DNA molecule is subjected may alter this probability. For example, increasing the external force could achieve a probability close to 1. The external force can be increased by increasing the magnitude of the E field, increasing the drag tag charge (q), or increasing the total number of molecules constituting the drag tag. The external force may also be decreased by decreasing these parameters. However, the efficacy of the drag strategy in achieving a particular orientation depends on the nature of the DNA molecule. Contrary to the results in FIGS. 1B and 1D, the dsDNA can achieve better alignment with the field lines due to its rigid nature and its low fluctuations. On the other hand, the highly fluctuating ssDNA requires a substantially larger force to achieve alignment. This is evident by the low probability of small markers in FIG. 2D (small number of tag molecules).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for manipulating a conformation and/or an orientation of a DNA molecule, the method comprising:
obtaining a DNA molecule;
modifying an end of the DNA molecule by coupling a charged molecule to the end of the DNA molecule;
applying an external force to the DNA molecule; and
adjusting a strength of the external force to induce the DNA molecule to change the conformation and/or the orientation of the DNA molecule.

2. The method of claim 1, wherein applying an external force to the DNA molecule includes subjecting the DNA molecule to an electric field.

3. The method of claim 2, wherein adjusting the strength of the external force includes adjusting the strength of the electric field.

4. The method of claim 2, wherein adjusting the strength of the external force includes adjusting the charge of the charged molecule.

5. The method of claim 1, wherein the charged molecule is a polyanionic tag.

6. The method of claim 1, wherein the charged molecule is a polycationic tag.

7. The method of claim 1, wherein a charged molecule is coupled to each end of the DNA molecule.

8. The method of claim 1, further comprising coupling the charged molecule to the end of the DNA molecule indirectly via PCR, hybridization, or ligation of an oligonucleotide conjugated to the charged molecule that is reverse complementary to the DNA molecule.

9. A method for unfolding a DNA molecule comprising:
obtaining a DNA molecule;
modifying an end of the DNA molecule by coupling a charged molecule to the end of the DNA molecule; and
subjecting the DNA molecule to an electric field.

10. The method of claim 9, wherein the charged molecule is a polyanionic tag.

11. The method of claim 9, wherein the charged molecule is a polycationic tag.

12. The method of claim 9, wherein a charged molecule is coupled to each end of the DNA molecule.

13. The method of claim 9, wherein the DNA molecule is single stranded.

14. A method for orienting a DNA molecule comprising:
obtaining a DNA molecule;
modifying an end of the DNA molecule by coupling a polyionic tag to the end of the DNA molecule to create a pseudo-dipole state;
subjecting the DNA molecule to an electric field; and
adjusting a strength of the electric field to induce the DNA molecule to align with an axis of the electric field.

15. The method of claim 14, further comprising adjusting the charge of the polyionic tag to induce the DNA molecule to align with an axis of the electric field.

16. The method of claim 14, further comprising adjusting the total number of molecules constituting the polyionic tag to induce the DNA molecule to align with an axis of the electric field.

17. The method of claim 14, wherein the DNA is single stranded.

18. The method of claim 14, wherein the polyionic tag is a polyanionic tag.

19. The method of claim 18, wherein the polyionic tag is a dendrimer.

20. The method of claim 14, further comprising coupling the polyionic tag to the end of the DNA molecule indirectly via PCR, hybridization, or ligation of an oligonucleotide conjugated to a polyionic tag that is reverse complementary to the DNA molecule.
